(19) United States
(12) Patent Application Publication
(10) Pub. No.: US 2007/0225208 A1
(43) Pub. Date: Sep. 27, 2007

(54) Use of Fibrin for Separating Body Organs

(75) Inventor: Pierpaolo Graziotti, Pavia (IT)

Correspondence Address: DICKSTEIN SHAPIRO LLP, 1825 EYE STREET NW, Washington, DC, 20006-5403, US

(73) Assignee: HUMANITAS MIRASOLE S.P.A., Rozzano (IT)

(21) Appl. No.: 11/596,620

(22) PCT Filed: May 20, 2005
(86) PCT No.: PCT/IT05/00288

§ 371 Date: May 8, 2007

(30) Foreign Application Priority Data
May 27, 2004 (IT) PCT/IT04/00310

(51) Int. Cl. A61L 31/00; A61M 37/00

(52) U.S. Cl. 514/2; 604/19

(57) ABSTRACT

The present invention relates to the use of coagulant or gellifying substances for administration into body sites with the aim of obtaining the temporary separation of said organs. The invention also relates to a method for the separation of body sites and a related administration kit using said substances.

The present invention relates to the use of coagulant or gellifying substances for administration into body sites and, particularly, relates to the use of fibrin and a related administration kit.

Prostate carcinoma is currently the most frequently diagnosed neoplasia in males.

Treatment of the neoplasia provides various therapeutic possibilities depending on the clinical stage of the disease, the patient's age and psychological disposition.

Among the most common treatments, conformational radiotherapy has therapeutic efficacy in relation to the prostatic volume, the targeting criteria for the site to be irradiated, and the dose that can be administered. To date, the radiotherapy doses universally considered to be suitable for adequate treatment are in excess of 70 Gy (Gray). However, suitable doses in excess of 70 Gy may be achieved with difficulty since they are correlated with increased complications, such as actinic (radiation induced) rectosigmoids. Indeed, despite the conformational approach of said treatment, the rectum, considering its anatomical closeness, may be affected by the radiation and hence damaged.

Considering the above, the current limit to raising the radiotherapy dose is precisely the risk of rectal damage.

Accordingly, the technical problem at the heart of the present invention is contriving a system which allows the patient to be prepared to receive a suitable dose of radiation aimed at the prostate without incurring the above mentioned drawbacks.

Said problem is resolved by the specific use of coagulant or gellifying substances, a related administration kit and an administration method, as described in the annexed claims.

The concept at the heart of the present invention is that of temporarily separating the prostate from the rectum during the entire duration of radiotherapy treatment, so that the radiation directed to the prostate does not also have negative effects on the rectum.

Further characteristics and the advantages of the present invention will be better understood from the description below of an embodiment of the invention, which is merely given by way of non-limiting example, wherein:

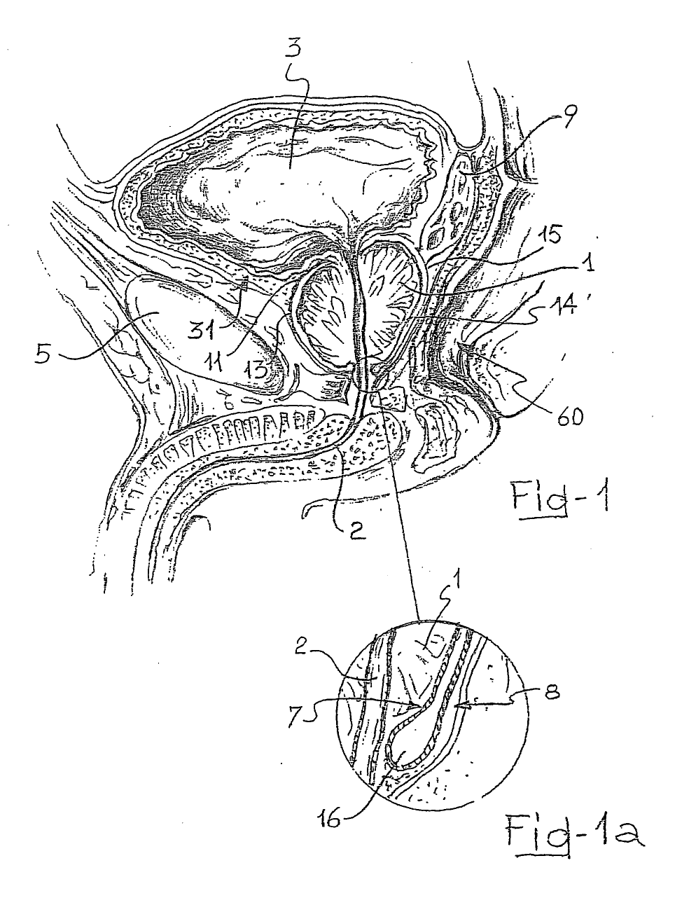

FIG. 1 represents a schematic midsagittal sectional view of human prostate and rectum relative to the adjacent organs;

FIG. 1a represents a detail of FIG. 1;

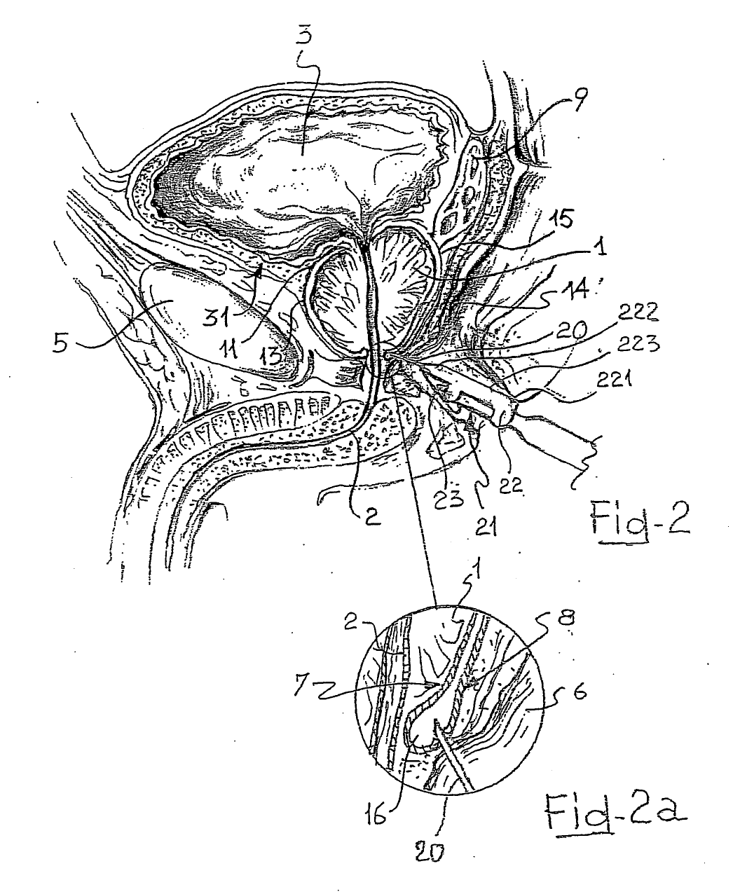

FIG. 2 represents a schematic view as in FIG. 1, during a first stage of the operation, in accordance with the invention;

FIG. 2a represents a detail of FIG. 2;

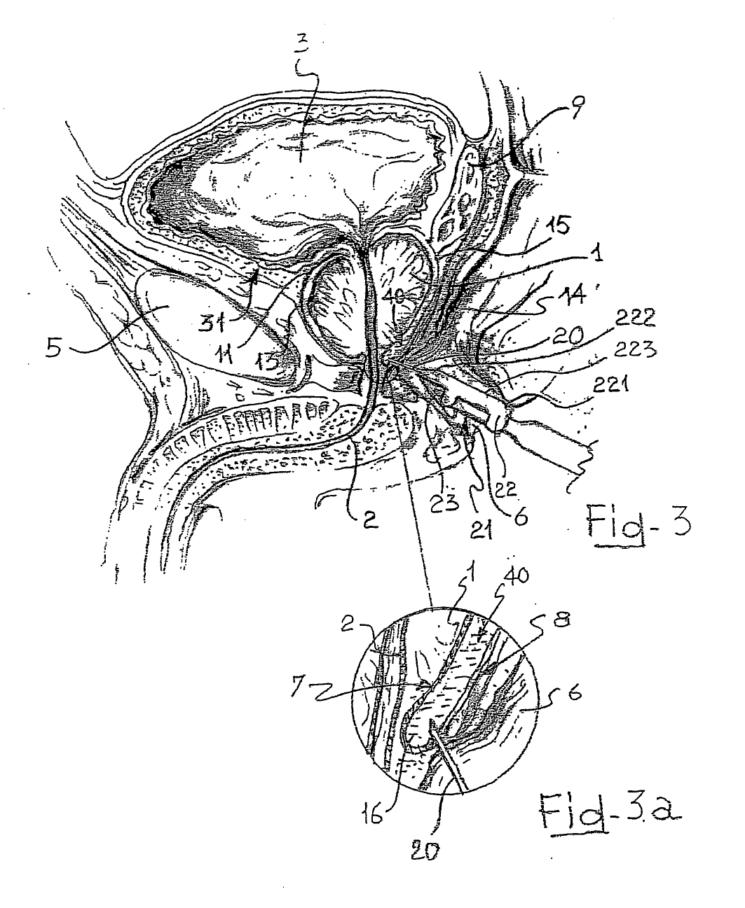

FIG. 3 represents a schematic view as in FIG. 1, during a second stage of the operation, in accordance with the invention;

FIG. 3a represents a detail of FIG. 3.

As represented in the figures, the prostate 1 is a glandular organ enclosing the initial tract of the male urethra 2 and has a conical-pyramidal shape with a larger base 11 and smaller apex 12, a front face 13, a rear face 14 and two side faces (not shown in the drawings).

The base 11 of the prostate 1 tightly adheres to the base 31 of the urinary bladder 3 forming the neck, while the apex 12 terminates just before the urogenital trigonal muscle 4.

The front face 13 is oriented towards the posterior side 51 of the pubic symphysis 5, while the rear face 14 is in close contact with the intestinum rectum (rectum) 6 through the interposition of the periprostatic fascia 7 and the recto-vesical fascia 8 (or Denonvilliers prostatoperitoneal fascia). Particularly, the periprostatic fascia 7 consists of a parietal sheet, in contact with and enclosing the prostate 1. At the apex 12, said fascia folds over on itself forming a sort of virtual space 16. In turns the rectovesical fascia 8 encloses the periprostatic fascia 7 and separates the rectum and prostate by arranging itself between the two organs. Sometimes, both fascias may be joined near the apex 12 of the prostate 1 creating said virtual space 16, as represented in FIGS. 1a, 2a and 3a.

The seminal vesicles 9 depart from the junction 15 between the base 11 and the rear face 14 of the prostate 1 (only one is shown in the figures) and extend towards the posterior on the outer surface of the base 31 of the urinary bladder 3.

The concept at the heart of the present invention of physically separating the prostate from the rectum by unattaching the rectovesical fascia from the periprostatic fascia and inserting a biocompatible substance between the two fascias, which may be reabsorbed after a suitable period of time.

Indeed, the exploitation of a substance having such characteristics as to create a reabsorbable separating wall or cushion, interposed between the prostate and the rectum has been considered. Thereby, the formation of a temporary inert physical space, allowing the targeted treatment of the prostate without also acting directly on the rectum, allows the resolution of the above mentioned problems.

FIGS. 2, 2a, 3 and 3a depict two stages of the method of administration of the biocompatible substance, allowing operation in the described manner.

In general, the method herein is a method for the separation of organs which are normally in contact, consisting of a stage with the administration of a coagulant or gellifying substance directly into a target site between said organs, such that said substance interposes itself between said organs and herein coagulates or gellifies, temporarily forming a dividing body.

Particularly, the patient is initially made to lie on one side with the knees folded on the chest (lateral decubitus) and a transrectal probe is applied, of the type conventionally used for echoradiographic monitoring of the prostate and the fascias thereof.

Preferably, a multiplanar transrectal ultrasound probe 21 equipped with a 7.0 MHz transducer (21 mm diameter at the apex, 24 mm at the base) as represented schematically in the figures, may be used. In this case, an appropriate needle 20, connected to a syringe (not shown) containing a substance adapted to the aforementioned purpose, is joined to the probe 21 by means of a guide 22. The guide 22 may be represented by any device suited to being reversibly applied to the probe 21 so as to allow the positioning of the tip of the needle 20 as close as possible to the end 23 of the probe itself.

Subsequently, the probe 21 is introduced into the anus 60 until its end 23 is close to the apex 12 of the prostate 1. Said position will be observed and monitored by the end itself which will be equipped with a standard echoradiographic detection system. Furthermore, the position of the tip of the needle 20 will be observed and followed, by means of a monitor connected to the probe.

Once the aforementioned position has been reached, the needle 20 is gently pushed against the rectal wall 6 until it penetrates through it, in the periapical area of the prostate 1, at its median line. As already mentioned, the advancement of the needle 20 is monitored by means of echography in order to be able to observe the moment in which the tip of the needle 20 reaches the plane delimited anteriorly by the periprostatic fascia 7 and posteriorly by the rectovesical fascia 8.

At this point, as depicted in FIG. 3, the biocompatible substance is injected into the virtual space 16 between said fascias. The injected fluid causes the progressive detachment of the two periprostatic 7 and rectovesical 8 fascias. Consequently, the prostate 1 is separated from the rectum 6 due to the interposition of said substance which, by being injected between said fascias until gradually reaching the seminal vesicles 9, forms a kind of pouch or bolus 40.

Alternatively, depending on particular requirements, the needle 20 may be introduced transperineally. By this means of access, still guided by means of echographic monitoring, the anatomical area of interest is equally reachable.

The transperineal alternative may be used in cases of high risk of infection associated with various clinical pathologies (diabetes, immunosuppression etc.) or in cases where the patient has a congenital or post-surgical anal inperforation. Indeed, it is obvious that the rectal wall is not sterile and may result in infections following manipulation.

It is understood that with the technique just described, both organs, i.e. the prostate 1 and the rectum 6, can be separated from each other, by a distance which may vary between 1 to 3 cm, allowing targeted irradiation of the prostate at appropriate doses while significantly limiting, if not even eliminating, irradiation of the anterior part of the rectum.

Particularly, as already mentioned, the substance injected to form the separating pouch 40 comprises any type of biocompatible substance capable of forming a gelatinous or solid coagulate or clot when injected into the target site. Preferably, this substance should have the capacity to be progressively reabsorbed by the body upon completion of treatment. Specifically, the treatment normally lasts up to 2 months.

The amount of substance to be injected may range between 10 and 40 ml, preferably between 20 and 30 ml depending on the anatomical characteristics of the patient and the physico-chemical characteristics of the substance itself. Generally, it has been observed that, on average, the amount used is 20 ml.

One particularly preferred example of this type of substance is represented by fibrin.

As is widely known, fibrin allows the formation of an entirely natural, and hence perfectly biocompatible coagulate, advantageously allowing itself to be gradually reabsorbed over time by the surrounding tissues, without causing any complications.

Fibrin may preferably consist of a formulation of its precursors such as fibrinogen and thrombin, in separate formulations. When the two separate formulations are injected into the target site, the corresponding precursors come into contact, mimicking the final stages of the blood coagulation cascade process, in order to form the well known coagulate, fibrin.

The fibrinogen formulation may comprise synthetic or naturally derived fibrinogen selected from commercially available sources. The amount may range between 40 mg/ml and 150 mg/ml, preferably ranging between 60 and 130 mg/ml, still more preferably between 70 and 110 mg/ml.

The thrombin formulation comprises synthetic or naturally derived thrombin from 250 UI to 2500 UI in amounts ranging between 15 mg/ml and 250 mg/ml, preferably ranging between 25 and 100 mg/ml, still more preferably between 50 and 70 mg/ml.

Additionally, the formulation may comprise antifibrinolytic ions and/or calcium ions in order to block or at least delay the fibrinolytic effect of endogenous substances such as plasmin.

The potential presence of Factor XIII in amounts ranging between 5 UI/ml and 50 UI/ml, preferably between 10 UI/ml and 20 UI/ml, may advantageously be useful for promoting or accelerating the polymerisation of fibrin monomers.

An additional component, allowing inhibition of the fibrinolytic action of plasmin, may be represented by the enzyme aprotinin. Aprotinin may be present in amounts ranging between 1.5 UPE/ml and 20 UPE/ml, preferably between 3.5 UPE/ml and 15 UPE/ml, still more preferably between 5 UPE/ml and 10 UPE/ml.

Thereby, the addition of said components to the fibrin precursors allows the delaying of the natural dissolution of the fibrin coagulate, and hence allows a more prolonged action over time.

Cited among those substances which may be preferably added to the fibrin-based solution, are fibronectin and plasminogen. Both such substances are well known components of the blood coagulation cascade, and for that reason may promote the formation of the coagulate in an entirely natural manner. Preferably, the fibronectin may be present in amounts ranging between 2 mg/ml and 30 mg/ml, preferably between 5 mg/ml and 20 mg/ml, still more preferably between 7 and 10 mg/ml. Instead, the amount of plasminogen may range between 0.02 mg/ml to 0.8 mg/ml, preferably from 0.08 mg/ml and 0.4 mg/ml, more preferably between 0.1 mg/ml and 0.2 mg/ml.

Obviously, the formulations may be prepared depending on the patients anatomical and physiological characteristics. Furthermore, the amounts may be adapted in accordance with the reabsorption times desired, which in turn will depend upon the radiotherapy treatment times. In any case, such adjustments are known by those skilled in the art.

Independently of the type of formulations used, it has been observed that in order to obtain a coagulate adapted to the previously described method, the fibrinogen must preferably have a multimeric, dimeric or trimeric structure, more preferably quadrimeric or quintomeric, still more preferably octameric or nonameric.

Certain products containing the above described substances as active ingredients are present on the market as fibrin glues. Fibrin glues are products which are considered to be scar-forming drugs, used in various medical sectors as adjuvants for stopping bleeds, for sealing tissues or for protecting sutures.

It has been discovered that the use of such substances or more generally, one or more biocompatible coagulation precursors (colloidal substances) for the preparation of one or more formulations which can be injected into body sites for in situ coagulate formation, is particularly effective in the separation of body organs.

It should be considered that in the present description, the term coagulation means any process which causes the transformation of a liquid into a gelatinous substance or a coagulate through the action of endogenous and/or exogenous chemical or physical agents. Consequently, the coagulate to which we are referring may be represented by the result of the coagulation of fibrin from its precursors, but may also refer to substances capable of gellifying, or rather giving rise to a coagulate in gel form.

One example of said substances which form a coagulate in gel form may be represented by collagen or the precursors thereof. Particularly, bovine collagen, autologous collagen, hyaluronic acid and polylactic acid may be cited. All such substances are already widely used in aesthetic medicine or in ocular surgery, hence they are highly biocompatible substances and have body reabsorption times comprised of between 60 days and 12 months.

With reference to collagen, the substance may be used in pure or mixed form. Preferably, the collagen is of type I, but either human or animal type II, III or IV may be used. Pure collagen is commercially available, for example under the brand names CosmoDerm® and CosmoPlast® produced by INAMED Aesthetics and containing human collagen. So-called mixed collagen is usually composed of a mixture of the aforementioned collagen types. Commercially available, mixed collagen based products are known, for example under the brand names Zyderm® (95% type I collagen and 5% type III collagen) at a concentration of 35 mg/ml (Zyderm® I, INAMED Aesthetics) and 65 mg/ml (Zyderm® II, INAMED Aesthetics). Alternatively, cross-linked collagen (Zyplast®) is also available. An additional substance which may be used is known by the brand name BioPlastique, a biodegradable biphasic copolymer (Bioplatique: A new textured copolymer microparticle promises permanence in soft tissue augmentation, Robert A. Ersek et al., Plastic and Reconstructive Surgery, April 1991, 693-702).

In relation to both natural and synthetic hyaluronic acid, its use has been known for some time, above all in the cosmetics industry, with the commercially available products Restylane, Perlane, Restylane Touch s and Restylane SUB-Q (Q-Med).

In relation to fibrin and derivatives thereof, various products are commercially available, known as the fibrin glues, included among which are, for example, TISSUCOL®, TISSUCOL® DUO and TISSEEL® VH available from Baxter, BERIPLAST® available from Aventis, TACHOCOMB® and TACHOCOM® H available from Nycomed Pharma or QUIXIL® available from Omrix Biopharmaceuticals Inc.

The substances in question may be sold in kit form for administration as illustrated above.

Particularly, the kit comprises one or more containers adapted to separately storing one or more biocompatible coagulation precursors, a syringe with corresponding needle suitable for the injection of said precursor(s) into the target site, a sterile disposable guide to be applied to a transrectal probe for guiding said needle into the target site and an information sheet explaining the use of the kit.

Precursors may be represented by fibrinogen and thrombin. Preferably, the fibrinogen and thrombin are stored in suitable containers in the form of ready-to-inject formulations. Alternatively, said substances may be stored in their corresponding containers in lyophilised form, and only dissolved in solution immediately prior to use. In this case, the kit will additionally comprise containers containing a physiological liquid capable of dissolving the lyophilisates, possibly with the aid of agitation.

Additionally, the kit may comprise one or more substances from among those previously described, each in an appropriate container, which may be used to promote the coagulation process and/or avoid the rapid degradation of the fibrin once the fibrinogen and thrombin have been injected into the target site. In this case, the kit may also comprise sterile disposable pipettes or syringes to allow the mixing or the preparation of the corresponding fibrinogen and thrombin formulations with appropriate or preferred substances, such as those described above.

The syringe for the injection of the fibrin and thrombin solutions may consist of a dual cylinder and dual plunger syringe or more simply a syringe having a body adapted to housing the two containers of the ready to use fibrinogen and thrombin formulations and two corresponding plungers.

The needle which may be applied to the syringe may be single barrelled or double barrelled—like a shotgun. In any case, the needle must have sufficient length to permit it to reach the site of diffusion. Preferably, said needle has a length of between 30 cm and 20 cm, more preferably between 20 cm and 15 cm, still more preferably comprised of between 10 and 5 cm. In addition, at least the tip of the needle must obligatorily have echoreflectance characteristics in order to allow the operator to constantly monitor its position while it is being introduced into the patient. For example, the echoreflectance may be provided by notches formed on the outer surface of the needle.

A needle possessing such characteristics is commercially available and known as the Chiba needle. Particularly, the Chiba needle, such as that sold under the trade name Ago Temno (Temno Chiba Fine Needle Aspiration, REF CHI2220 22G×20 cm) by Allegiance Healthcare Corporation, has a mandrel incorporated inside the needle barrel. Particularly, the mandrel is fitted with flared ends so as to close the corresponding flared bore of the barrel.

Typically, the Chiba needle is 20 cm long and is used for performing local anaesthesia prior to echo-guided prostate biopsy operations. In this case, the mandrel might be removed from the needle in order to allow injection of the coagulant substance or gel.

Alternatively, it is possible to arrange for a needle fitted with two matched barrels which are joined together only at the distal part such as to allow the mixing of the coagulant substance or gel precursors as close to the target site as possible. In this case, any premature coagulation or gelification inside the barrel of the needle which might result in the blockage of the needle itself, is avoided.

Furthermore, other commercially available disposable needles may be used according to any particular requirements or substances used. In any case, whatever type of needle is used, it is necessary for it to be echoreflective.

The needle guide may be a device such as that represented in the figures. Particularly, such device 22 comprises a distal end 221 connected to a proximal end 222 by means of a bridge 223. The proximal 221 and distal 222 ends are made such as to be reversibly coupled to a transrectal probe, such as that previously described. Preferably, said parts are shaped as part of a circular band which snaps onto the end of said probe. The bridge 223 besides connecting said ends, is also used to hold the needle. Indeed, a straight channel is accommodated therein, open at both ends, so as to allow for example, the insertion of the Chiba needle into the proximal end with its protrusion from the distal end.

Preferably, the kit also comprises one or more sterile disposable condoms, for fitting over the end section of a transrectal probe.

The information sheet should contain all the useful information for the description of the kit components and the storage thereof. The preparation methods for the solutions and the manipulation thereof prior to application, as described previously for example, will also have to be specified-. Finally, the information sheet must contain an explanation of the assembly of the syringe, precautions for the use thereof, any contraindications relating to the substances included and, preferably, a clear illustration of the method of administration.

From the description, it should be appreciated that, the use of a substance such as fibrin and the related kit, satisfies the requirements reported in the introduction to the present invention.

Obviously, one skilled in the art, in satisfying contingent and specific needs, may introduce alterations or embodiments to the use of the above described kit, all however contemplated by the invention as defined in the following claims.

1. Use of a biocompatible coagulant or gellifying substance for the preparation of a formulation that may be injected into body sites for the in situ formation of a solid or gelatinous coagulate, with the aim of separating two or more body organs from one another.

2. The use according to claim 1, wherein said substance is fibrin.

3. The use according to claim 2, wherein the fibrin consists of a composition comprising at least fibrinogen and thrombin, the two precursors thereof.

4. The use according to claim 3, wherein said fibrin-based composition comprises amounts of fibrinogen ranging between 40 mg/ml and 150 mg/ml and amounts of thrombin ranging between 15 mg/ml and 250 mg/ml.

5. The use according to claim 3, wherein said composition comprises antifibrinolytic agents.

6. The use according to claim 5, wherein said antifibrinolytic agents comprise amounts of antifibrinolytic ions and/or aprotinin ranging between 1.5 UPE and 20 UPE.

7. The use according to claim 3, wherein said composition comprises a natural or synthetic factor for promoting the polymerisation of the fibrin monomers.

8. The use according to claim 7, wherein said factor is selected from the group consisting of Factor XIII, fibronectin and fibrinogen, possibly in combination with one another in amounts ranging between 5 UI/ml and 50 UI/ml for Factor XIII, between 2 mg/ml and 30 mg/ml for fibronectin, between 0.02 mg/ml and 0.8 mg/ml for plasminogen.

9. The use according to claim 3, wherein the fibrinogen has a multimeric structure.

10. The use according to claim 9, wherein said fibrinogen multimeric structure is dimeric, trimeric, quadrimeric, quintomeric, octameric or nonameric.

11. The use according to claim 1, wherein said substance is a fibrin-based glue.

12. The use according to claim 11, wherein said fibrin glue is selected from the products TISSUCOL, TISSUCOL DUO, TISSEEL VH, BERIPLAST, TACHOCOMB, TACHOCOM and QUIXIL.

13. The use according to claim 1, wherein the biocompatible coagulant or gellifying substance is selected from collagen and the precursors thereof, hyaluronic acid and polylactic acid.

14. The use according to claim 13, wherein the collagen is pure type I, II, III or IV and mixtures thereof, both of animal and human origin.

15. The use according to claim 13, wherein the collagen is selected from the commercially available products CosmoDerm, CosmoPlast, Zyderm, Zyplast.

16. The use according to claim 13, wherein the hyaluronic acid is either synthetic or natural, preferably selected from the commercially available products Restylane, Perlane, Restylane Touch S and Restylane SUB-Q.

17. The use according to claim 1, wherein the coagulant or gellifying substance is the biphasic copolymer BioPlastique.

18. A kit for the administration of a biocompatible coagulant or gellifying substance into body sites, comprising containers adapted to separately storing fibrinogen and thrombin, a syringe adapted to simultaneously injecting fibrinogen and thrombin into the target site, fitted with an appropriate needle, a guide adapted to guiding said needle into the target site and an information sheet explaining the use of the kit.

19. The kit according to claim 18, wherein said containers separately contain fibrinogen and fibrin in injectable formulations.

20. The kit according to claim 18, wherein said containers separately contain fibrinogen and fibrin in lyophilised form and, wherein said kit comprises at least one additional container containing a physiological liquid adapted to the preparation of an injectable solution, suspension or emulsion of said fibrinogen and fibrin.

21. The kit according to claim 18, additionally comprising at least one container for a substance capable of promoting the coagulation process and/or slowing down the natural degradation of fibrin.

22. The kit according to claim 18, additionally comprising sterile disposable pipettes or syringes.

23. The kit according to claim 18, wherein said syringe for the injection of said fibrinogen and thrombin solutions comprises two cylinders and two plungers.

24. The kit according to claim 23, wherein said syringe has a single barrel.

25. The kit according to claim 23, wherein said syringe is a double barrelled syringe.

26. The kit according to claim 24, wherein said needle is an echoreflective needle.

27. A kit for the administration of a biocompatible coagulant or gellifying substance into body sites, comprising containers suitable to store a gellifying substance in accordance with claim 13, a syringe adapted to injecting said substance into the target site, fitted with an appropriate needle, a guide adapted to guiding said needle into the target site and an information sheet explaining the use of the kit.

28. A method for separating body organs comprising a stage with the administration of a coagulant or gellifying substance directly into a target site such a that said substance interposes itself between said organs and herein coagulates or gellifies, thus forming a kind of partitioning wall.

29. The method according to claim 22, wherein said administration stage is carried out using a syringe.

30. The method according to claim 28, wherein said administration is provided between the prostate and the rectum in order to separate said organs from one another.

31. The method according to claim 30, wherein said administration is provided between the periprostatic fascia and the rectovesical fascia, by passing through the rectal wall.

32. The method according to claim 31, wherein said administration is performed by injecting said substance, starting from the prostate apex until reaching the seminal vesicles.

33. The method according to claim 28, wherein said substance is injected such as to separate the prostate and the rectum by a distance ranging between 2 and 4 cm.

34. The method according to claim 28, wherein said substance is a fibrin, collagen, collagen precursor, hyaluronic acid or lactic acid based composition.

35. The method according to claim 34, wherein said substance is injected in quantities ranging between 20 and 30 ml.

36. The method according to claim 28, wherein said syringe is provided with an echoreflective needle, the position of which is monitored by means of specific echography.

37. The method according to claim 36, wherein said needle is a Chiba needle.

38. The method according to claim 28, wherein said substance is fibrin.

39. The method according to claim 28, wherein said administration is made transperineally.
